(19) United States
(12) Patent Application Publication
(10) Pub. No.: US 2024/0043373 A1
(43) Pub. Date: Feb. 8, 2024

(54) COMPOSITIONS AND METHODS FOR TREATING AMYLOID-RELATED CONDITIONS

(71) Applicant: The Regents of the University of California, Oakland, CA (US)

(72) Inventors: Varghese John, Los Angeles, CA (US); Jesus J. Campagna, Playa Del Rey, CA (US); Sujyoti Chandra, Los Angeles, CA (US); Samuel W. Johnson-Cohn, Los Angeles, CA (US); Barbara Jagodzinska, Los Angeles, CA (US)

(21) Appl. No.: 18/023,594

(22) PCT Filed: Aug. 27, 2021
(86) PCT No.: PCT/US21/47894

§ 371 Date: Feb. 27, 2023

Related U.S. Application Data
(60) Provisional application No. 63/071,640, filed on Aug. 28, 2020.

(51) Int. Cl. C07C 237/20; A61P 25/28

(52) U.S. Cl. CPC C07C 237/20; A61P 25/28; C07C 2601/16

(57) ABSTRACT

The present disclosure relates to compounds that are capable of upregulating sAPPα and/or stabilizing reticulon-3. The disclosure further relates to methods of treating neurodegenerative diseases and disorders (e.g., Alzheimer's disease).

### RELATED APPLICATIONS

This application claims the benefit of U.S. Provisional Application No. 63/071,640 filed Aug. 28, 2020, the contents of which are fully incorporated by reference herein.

### STATEMENT OF GOVERNMENT SUPPORT

This invention was made with government support under Grant Number AG052195, awarded by the National Institutes of Health. The government has certain rights in the invention.

### BACKGROUND

Alzheimer's disease (AD), which currently affects ˜6 million Americans—a number that is predicted to increase to 14 million by 2050—is a progressive neurodegenerative disorder characterized by the presence of senile plaques composed mainly of amyloid β-protein (Aβ), and the development of neurofibrillary tangles resulting from the hyper-phosphorylation of microtubule-stabilizing protein tau in brain tissue. In AD, impairment of cholinergic transmission starting in the nucleus basalis of Maynert contributes to cognitive decline, and thus has been a target for therapeutic intervention. The currently available FDA-approved treatments for AD are acetylcholinesterase inhibitors or antagonist of the NMDA receptor. The clinical benefits of these drugs are modest, temporary and do not specifically target the cellular mechanisms of AD including generation of neurotoxic Aβ, p-tau or apolipoprotein ε4 (ApoE4)-related changes that precipitate onset of the disease. Thus, there is an unmet ongoing need for new treatments of AD.

### SUMMARY OF THE INVENTION

In one aspect, the present disclosure provides compounds of formula I or a pharmaceutically acceptable salt thereof:

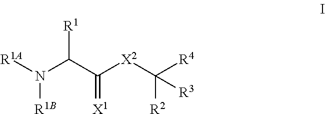

wherein
X¹ and X² are each independently NR^{2A}, O, or S;
R¹ is a side chain of a natural amino acid, alkyl, hydroxyalkyl, alkyloxyalkyl, aminoalkyl, thioalkyl, cycloalkyl, heterocyclyl, aryl, heteroaryl, aralkyl, or heteroarylalkyl;
R² and R⁴ are each independently cycloalkyl, heterocyclyl, aryl, heteroaryl, aralkyl, or heteroarylalkyl;
R³ is H, alkyl, hydroxyalkyl, alkyloxy, alkyloxyalkyl, aminoalkyl, or thioalkyl;
R^{1A}, R^{1B}, and R^{2A} are each independently selected from H, alkyl, and aralkyl.

In another aspect, the present disclosure provides methods of treating neurodegenerative diseases and disorders.

### BRIEF DESCRIPTION OF THE DRAWINGS

FIG. 1 depicts the amyloid precursor protein (APP) processing cycle. Full-length APP (FL APP) can be sequentially cleaved by BACE1 and γ secretase to form plaque-associated amyloid-β (Aβ)., Alternatively, FL APP can be cleaved by α secretase ADAM10 for form trophic, neurite- and neuronal plasticity-support fragment sAPPα—the target for upregulation and enhancement.

FIG. 2 depicts that sAPPα inhibits BACE1 and reduces β-CTF.

FIG. 3 depicts the correlation of sAPPα to cognition. Raw block design scores are plotted against sAPPα.

FIG. 4 shows that sAPPα mutation leads to AD. The k16N APP mutation leads to both early-onset AD and lowering of both sAPPα and αCTF.

FIG. 5A shows HMW RTN3 oligomer formation in the AD afflicted brain. 13 of 18 AD frontal cortex samples had HMW oligomers (circled) in contrast to 1 of 10 in non-demented (ND) brains.

FIG. 5B shows the quantification of RTN3 monomers showed that the monomers are significant higher in the ND brain as compared to the AD brain.

FIG. 6A decpits a proposed model of the effect of RTN3 on sAPPα formation. Following synthesis in the ER, BACE 1 is transported through the golgi to the cell surface and endosome. Interaction of BACE1 with APP generates Aβ in the endosome.

FIG. 6B shows that in the presence of increased RTN3 mono, RTN3 retains BACE1 in the ER thereby antagonizing BACE1 transported to the endosome. Accordingly, the generation of Aβ is reduced and the generation of non-amyloidogenic APP-cleavage product sAPPα is increased.

FIG. 7 shows the structures of DDL-401 and DL-402.

FIG. 8 shows the effects of DLL-401 in SH-SY5Y cells. Different doses (5, 10, 50 uM) of DDL-401 increased sAPPα in SH-SY5Y cells (150 K/well) treated for 24 hours. *p<0.05, **p<0.01, ***p<0.001.

FIG. 9 shows the effects on RTN3 monomer after 24 h treatment with DLL-401 in SH-SY5Y cells plated at 400,000/well . The level of RTN3 monomer increases with an escalating DDL-401 dose.

FIGS. 10A & B show the PK of DDL-401. Mice were dosed at 10 mg/kg either subcutaneously (sc) or orally and brain/plasma levels of DDL-401 were determined 1, 2, 4, and 8 hours later. The sc brain peak (approximately 1640 ng/g) was observed at 1 hour and the oral peak (approximately 63 ng/g) was observed at 2 hours.

FIG. 11 is an illustration of the SAR strategy for developing analogs of DDL-401.

FIG. 12A is graph showing the time dependent increase in sAPPα following the administration of DDL-401. SH-SY5Y cells at 400,000/well were plated in a 12-well plate and treated with 5 μM (final concentration) of DDL 401 (top) or with DMSO (bottom). Media (20 μL) was collected every 2 h for 24 h (plate was shaken prior to media collection to disburse sAPPα) from each treatment group.

FIG. 12B shows that both DDL401 and DDL402 significantly increased sAPPα levels after 24 h.

FIG. 13 Structural determinants of the RTN3-BACE1 binding by crosslinking mass spectrometry. Key residues such as CLR and HDD that have been previously reported to be involved in the binding are shown by dashed arrow. The interaction of RTN3 with BACE prevents BACE cleavage of APP.

FIGS. 14A & B show that treatment with DDL-401 and DDL-402 in SH-SY5Y cells shows trend to increase in p-GSK3β (Ser 9). SHSYSY cells (100 k/well in 24 well plate) were treated with 5 uM Dilazep (DDL402) or 50 uM Remacemide (DDL401) for 18 h. FIG. 14A shows the levels of phospho GSK3β (p-GSK3β) and total GSK3β (T-GSK3b) which were analyzed by western blot. FIG. 14B is a graph showing each blot normalized to actin levels. The ratio of (phospho-GSK3β/Actin) to (Total-GSK3β/Actin) were calculated by densitometry and shows a trend to increase in p-GSK3β. The treatment with DDL-401 and 402 shows a trend to increase in p-GSK3β which is known to correlate to decrease in the p-tau production that leads to deposition of the tangle pathology in Alzheimer's and spread of the disease. This effect of DDL-401 & 402 may be due to its enhancement of sAPPalpha, which is known to increase p-GSK3β.

FIGS. 15A & B show that RTN3 levels trend to increase in SHSY5Y cells treated with sAPPα enhancer Tropisetron (F03). FIG. 15A shows SHSY5Y cells (100 k/well in 24 well plate) treated with 5 uM F03 for 12 h. RTN3 levels (monomer ˜25 kD) were analyzed by western blot. FIG. 15B shows that the level of RTN3 monomer trended to increase in F03 treated cells compared to DMSO or untreated cells.

FIGS. 16A-C show that F03 increases phosphorylation of GSK3β in SHSY5Y cells. FIGS. 16A & B shows a Western blot where SHSY5Y cells (100 k/well in 24 well plate) were treated with 5 uM F03 or recombinant sAPPα (25 nM) for 12 h or 18 h. sAPPα has been shown to increase the phosphorylation of GSK3β (Deng et al., 2015), hence recombinant sAPPα was used as a positive control. FIG. 16C is a graph decpiting the normalized to actin levels and the ratio of (phosphor-GSK3β/Actin) to (Total-GSK3β/Actin) from the Western blots in FIGS. 16A & B, calculated by densitometry.

FIGS. 17A-C show that the treatment of ApoE4:5xFAD (E4:FAD) mice with F03 effects RTN3, sAPPα and p-GSK3β. FIG. 17A shows that RTN3 levels can be slightly enhanced in the brain after treatment with a small molecule F03 orally at 4 mg/kg for 18 days. FIG. 17B shows that there is a concomitant slight increase in sAPPα and FIG. 17C shows a bigger increase in p-GSK3β in brain after F03 in vivo treatment. This data suggest that RTN3 modulators, can affect the neurotrophic protein sAPPα levels, and can also modulate the p-tau levels and tangle pathology in Alzheimer's disease through its effects on p-GSK3β and thus can ultimately suppress the spread of the disease.

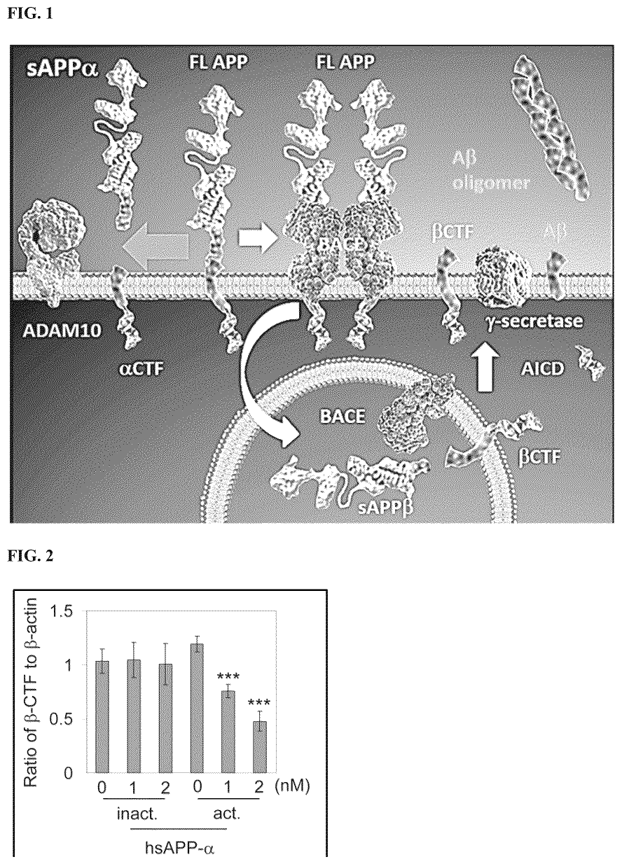

### DETAILED DESCRIPTION OF THE INVENTION

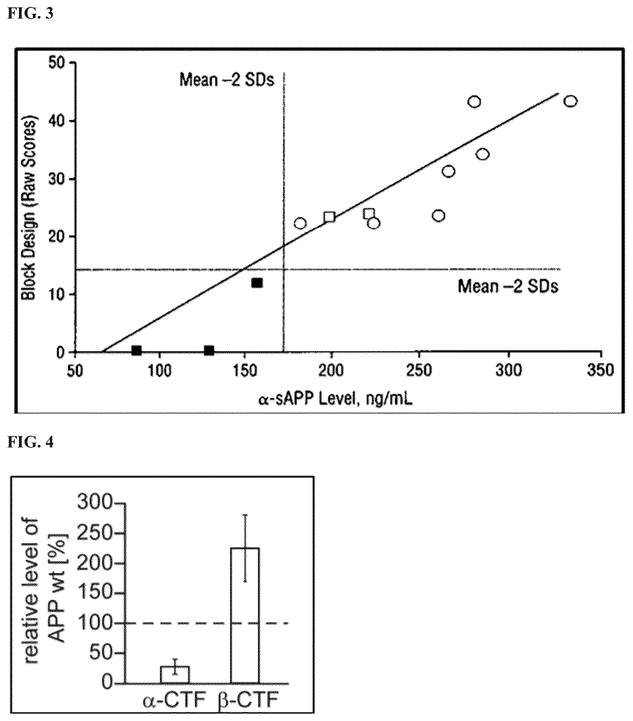

The pathological hallmarks of AD are neuritic plaques comprised largely of amyloid beta peptide (Aβ) and neurofibrillary tangles resulting from tau microtubule protein hyperphosphorylation. Aβ originates from amyloid precursor protein (APP) which may be cleaved via alternate pathways (FIG. 2). In the anti-trophic, pro-AD pathway, β-secretase (BACE1) interacts with APP on the cell surface, leading to dimerization and endocytosis to an acidic compartment wherein it is cleaved to produce sAPPβ and βCTF. The βCTF fragment then can undergo γ-secretase cleavage, generating Aβ of a variety of species (lengths). Alternatively, APP can be cleaved by the α-secretase the disintegrin and metalloprotease ADAM10 to generate two fragments sAPPα and αCTF. These latter trophic, anti-AD fragments support synaptic maintenance and can inhibit the β-pathway. sAPPα itself is a BACE1 inhibitor (FIG. 3) and CTF can inhibit γ-secretase activity. Support for ADAM10 as the likely α-secretase is based on the missense mutation in ADAM10 that potentiates Aβ accumulation while decreasing the sAPPα/sAPPβ ratios and overexpression of ADAM10 results in reversal of the AD pathology in mice.

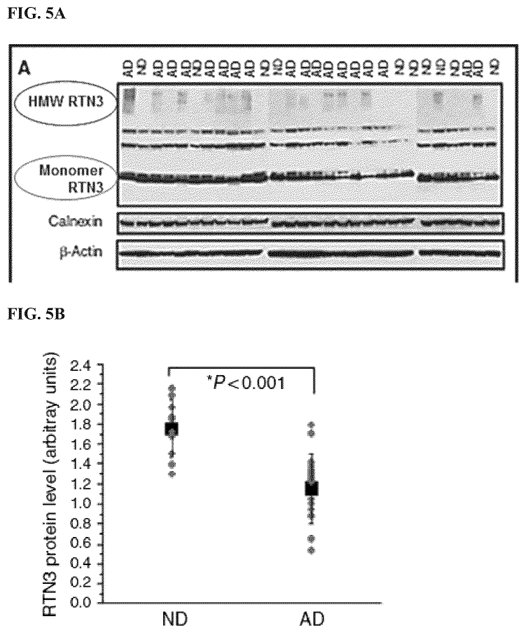

The anti-AD functions of sAPPα as revealed by in vitro and in vivo studies include direct inhibition of the BACE1 enzyme, inhibition of tau phosphorylation via the GSK3β pathway, reduction of excitability (in primary neurons), and ability to increase synaptic elements. Multiple studies have reported on the pro-cognitive functions of sAPPα. The role of sAPPα in synaptic plasticity and maintenance is well established in vivo. For example, positive correlation has been shown between performance in spatial memory tasks and CSF sAPPα levels in young and aged rats. ICV treatment of mice with sAPPα improved both motor and cognitive function in mice subjected to traumatic brain injury (TBI), another pathological condition resulting in increased β pathway processing of APP. It has also been shown that acute sAPPα administration can rescue LTP in conditional APP/APLP knockout mice. In humans, CSF sAPPα levels correlate positively with better cognitive performance as determined by IQ, verbal ability, visuospatial function, immediate memory, episodic memory and various aspects of attention (FIG. 4) and levels of sAPPα is decreased in patients carrying the major AD risk factor, ApoE4. Conversely, a mutation at the α-secretase cleavage site was shown to lead to decreased sAPPα (FIG. 5) and early-onset AD.

Initial HTS led to identification of the compound Remacemide as an enhancer of sAPPα in Chinese Hamster ovary (CHO) cells that stably express human APP (CHO-7W cells) and the effects were confirmed in human neuroblastoma SH-SY5Y cells (Example 1). Global proteomic analysis of drug-treated neurons revealed this novel mechanism-of-action (MOA) involving enhancement of sAPPα through modulation of a protein reticulon-3 (RTN3).

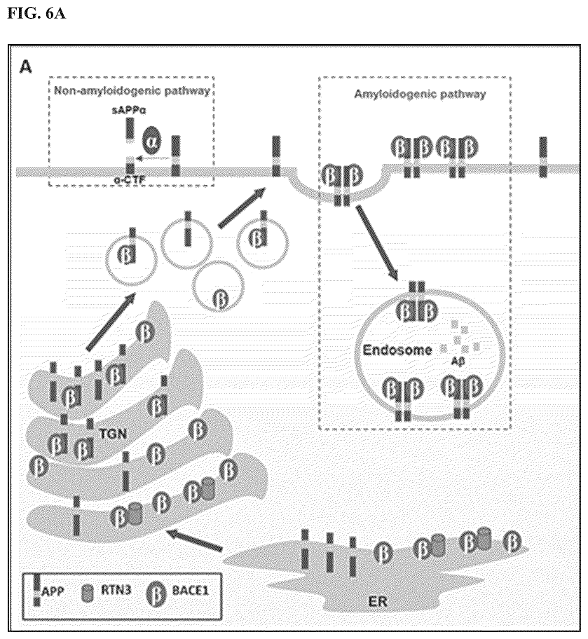

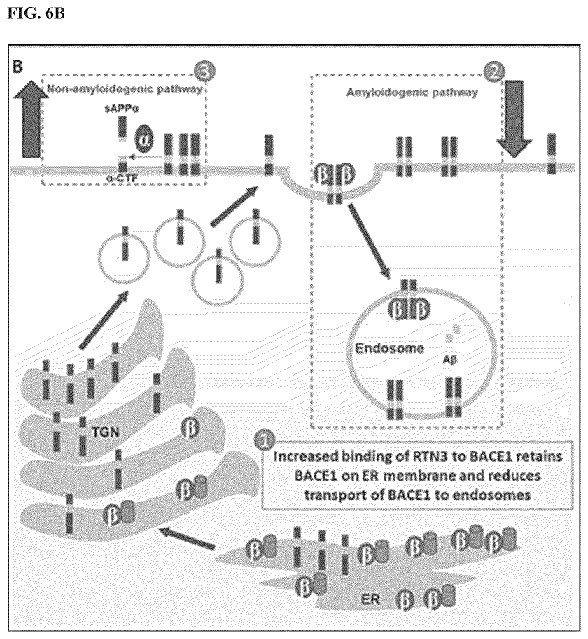

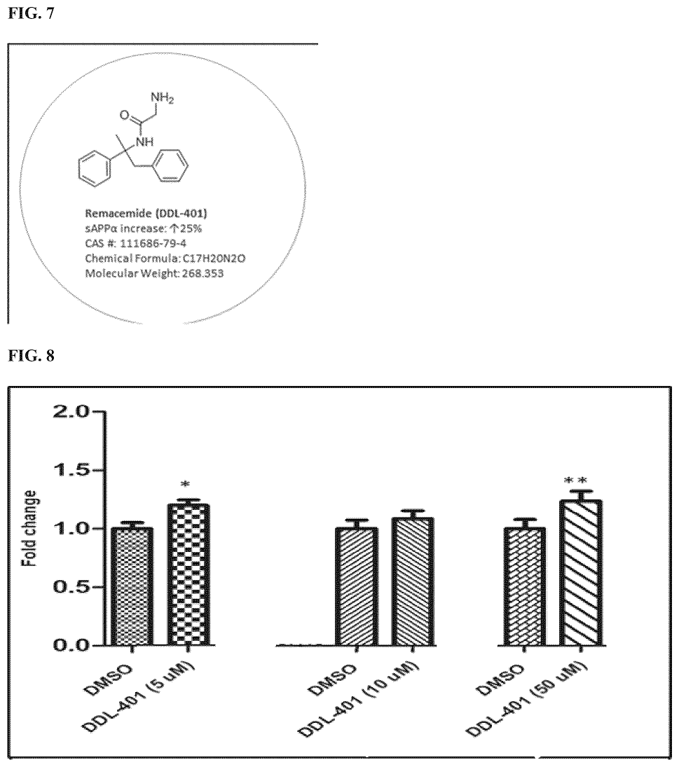

RTN3 is a highly neuronally-expressed reticulon protein, and RTN3 monomers have been shown to negatively regulate BACE1; there is also biochemical and morphological evidence that supports a role of RTN3 in formation of amyloid plaques. In AD patients, high molecular weight (HMW) oligomers of RTN3 were found to be increased in frontal cortex of the AD brain tissue as compared to non-demented (ND) controls (FIG. 6A) and, conversely, levels of RTN3 monomers were found to be significantly reduced in AD brain (FIG. 6B). RTN3 deficiency was recently reported to increase Aβ plaque load due to a reduction of RTN3s negative modulation of BACE1 activity. RTN3 monomers can directly bind to BACE1, preventing its interaction with APP. Additionally, RTN3 retains BACE1 in the ER, limiting the pool of BACE1 in the acidic endosomes wherein amyloidogenic cleavage occurs (FIG. 7). Transgenic mice overexpressing RTN3 in neurons show reduced amyloid deposition, while knocking out RTN3 in mice leads to enhanced amyloid deposition. More recently, four RTN3 variants were reported to be potential risk factor for AD. In human AD and transgenic mouse model brain tissue, RTN3—if destabilized—forms aggregates in dystrophic neurites, thus preventing negative regulation of BACE1 by RTN3. The C-terminal region of RTN3 is required to bind to BACE1, which mainly interacts with RTN3 monomer. HM cells were transfected with RTN constructs Ax-RTN3 (full length) and R3-ΔC36 (missing 36 c-terminus amino acids) either alone or together for 48 h and the lysates were prepared for immunoprecipitation with the anti-HA matrix. RTN3 variants on the blot were detected with Xpress antibody. The C-terminal region of BACE1 is critical for the binding to RTN3. HR3M cells were transfected with a series of BACE1 deletion constructs for 48 h and protein extracts were used for Western blot analysis after immunoprecipitation with the myc antibody 9E10 recognizing RTN3. Antibody B279 recognizing BACE1 residues 295 to 310 was used for detection. Mutants missing at least 17 c-terminal amino acids (B1-QWR, B1-CX, B1-ATMC-KDEL) suppress BACE1 binding to RTN3. RTN3 blocks BACE1 interactions with APP. 125.3 cells were transfected with the indicated constructs for 48 h, and cell lysates were subjected to immunoprecipitation with anti-HA affinity matrix. APP was detected with 6E10 monoclonal antibody, BACE1 variants were detected with anti-HA, RTN3 was detected 9E10 antibody to Myc. Increasing expression of RTN3 correspondingly increased coimmunoprecipitation of BACE1 D92G—HA with RTN3, but reduced coimmunoprecipitation of BACE1 D92G-HA with APP. Given the extremely important role of RTN3 in Alzheimer's disease, drug-mediated increase in RTN3 monomer and thus inhibition of BACE and increase in the non-amyloidogenic APP cleavage product sAPPα represents a therapeutic strategy for the treatment of AD.

In one aspect, the present disclosure provides compounds of formula I or a pharmaceutically acceptable salt thereof:

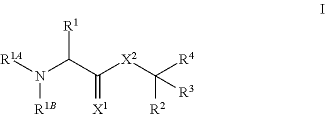

wherein
X¹ and X² are each independently NR^{2A}, O, or S;
R¹ is a side chain of a natural amino acid, alkyl, hydroxyalkyl, alkyloxyalkyl, aminoalkyl, thioalkyl, cycloalkyl, heterocyclyl, aryl, heteroaryl, aralkyl, or heteroarylalkyl;
R² and R⁴ are each independently cycloalkyl, heterocyclyl, aryl, heteroaryl, aralkyl, or heteroarylalkyl;
R³ is H, alkyl, hydroxyalkyl, alkyloxy, alkyloxyalkyl, aminoalkyl, or thioalkyl;
   R^{1A}, R^{1B}, and R^{2A} are each independently selected from H, alkyl, and aralkyl.

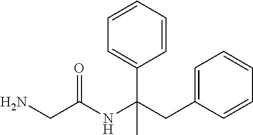 [SMILES: CC(Cc1ccccc1)(NC(=O)CN)c1ccccc1]

In certain embodiments of formula I, the compound is not or a pharmaceutically acceptable salt thereof.

In certain embodiments of formula I, if X¹ is O, X² is NH, R¹ is H (i.e., the side chain of glycine), R² is phenyl, R³ is methyl, R⁴ is benzyl, and R^{1A} and R^{1B} are both H, then R² is substituted. In other embodiments of formula I, if X¹ is O, X² is NH, R¹ is H (i.e., the side chain of glycine), R² is phenyl, R³ is methyl, R⁴ is benzyl, and R^{1A} and R^{1B} are both H, then R⁴ is substituted.

In certain embodiments of formula I, R¹ is the side chain of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine. In certain embodiments, R¹ is the side chain of valine, leucine, phenylalanine, isoleucine, phenyl, norleucine, or glycine.

In certain embodiments of formula I, X¹ is O.

In certain embodiments of formula I, X² is NR^{2A}.

In certain embodiments of formula I, R² is aryl or heteroaryl. In certain embodiments, R² is substituted with or more R⁵; and each R⁵ is independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, heterocyclylalkyl, heteroaralkyl, sulfonamide, aryl, heteroaryl, heterocyclyl, and aralkyl. In certain embodiments, R² is substituted with or more R⁵; and each R⁶ is independently selected from alkyl (e.g., trifluoromethyl) or halo (e.g., fluoro). In certain embodiments, R² is substituted with 1, 2, 3, 4, or 5 R⁵, preferably 1 or 2 R⁵. In certain preferred embodiments, R² is aryl (e.g., 3,4-difluorophenyl, 2,4-difluorophenyl, 4-trifluoromethylphenyl). In certain embodiments, R² is heteroaryl (e.g., pyridyl, 2,4-difluoropyridyl, pyrimidinyl). In certain embodiments, R² is a five membered heteroaryl heteroaryl.

In certain embodiments of formula I, R³ is alkyl (e.g., methyl).

In certain embodiments of formula I, R⁴ is aralkyl (e.g., benzyl), aryl (e.g., phenyl), heteroaryl (e.g., pyridinyl or benzothiazolyl), or heterocyclyl (e.g., indolinyl). In certain embodiments, R⁴ is substituted with or more R⁶; and each R⁶ is independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, heterocyclylalkyl, heteroaralkyl, sulfonamide, aryl, heteroaryl, heterocyclyl, and aralkyl. In certain preferred embodiments, R⁴ is substituted with or more R⁶; and each R⁶ is independently selected from alkyl (e.g., methyl), aryl (e.g., phenyl), or halo (e.g., fluoro). In certain embodiments, R⁴ is substituted with 1, 2, 3, 4, or 5 R⁶, preferably 1 or 2 R⁶. In certain embodiments, R⁴ is aralkyl (e.g., benzyl, 4-fluorobenzyl, 2,4-difluorobenzyl, or dimethylbenzyl). In certain embodiments, R⁴ is aryl (e.g., phenyl or biphenyl). In certain embodiments, R⁴ is heteroaryl (e.g., pyridinyl or benzothiazolyl). In certain embodiments, R⁴ is heterocyclyl (e.g., indolinyl).

In another aspect, the present disclosure provides methods of treating a neurodegenerative disease or disorder in a subject in need thereof, comprising administering a compound of formula I or a pharmaceutically acceptable salt thereof to the subject. In certain embodiments, the neurodegenerative disease or disorder is Alzheimer's disease.

In another aspect, the present disclosure provides methods of treating a neurodegenerative disease or disorder in a subject in need thereof, comprising administering a compound to the subject, wherein the compound is

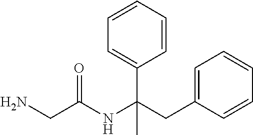 [SMILES: CC(Cc1ccccc1)(NC(=O)CN)c1ccccc1]

or a pharmaceutically acceptable salt thereof. In certain embodiments, the neurodegenerative disease or disorder is Alzheimer's disease.

In yet another aspect, the present disclosure provides methods of treating a neurodegenerative disease or disorder in a subject in need thereof, comprising administering an upregulator of sAPPα to the subject. In certain embodiments, the upregulator of sAPPα is a compound of formula I or a pharmaceutically acceptable salt thereof. In other embodiments, the upregulator of sAPPα is

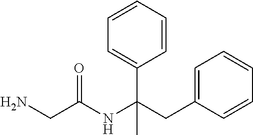 [SMILES: CC(Cc1ccccc1)(NC(=O)CN)c1ccccc1]

or a pharmaceutically acceptable salt thereof. In certain embodiments, the neurodegenerative disease or disorder is Alzheimer's disease.

In yet another aspect, the present disclosure provides methods of treating a neurodegenerative disease or disorder in a subject in need thereof, comprising administering a stabilizer of reticulon-3 (RTN3) to the subject. In certain embodiments, the stabilizer of RTN3 is a compound of formula I or a pharmaceutically acceptable salt thereof. In other embodiments, the stabilizer of RTN3 is

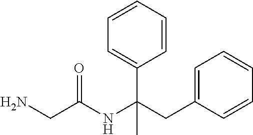 [SMILES: CC(Cc1ccccc1)(NC(=O)CN)c1ccccc1]

or a pharmaceutically acceptable salt thereof. in certain embodiments, the neurodegenerative disease or disorder is Alzheimer's disease, traumatic brain injury (TBI), stroke, age related macular degeneration (AMD) or amyotropic lateral sclerosis (ALS). In certain embodiments, the neurodegenerative disease or disorder is Alzheimer's disease.

In yet another aspect, the present disclosure provides methods of upregulating sAPPα and/or stabilizing RTN3 in a cell in vivo comprising contacting the cell with a compound of formula I or

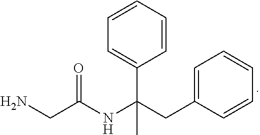 [SMILES: CC(Cc1ccccc1)(NC(=O)CN)c1ccccc1]

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid , naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

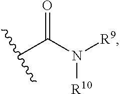

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

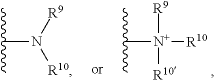

wherein R⁹, R¹⁰, and R¹⁰, each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

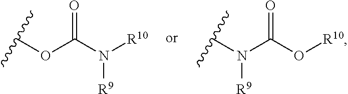

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, denzodioxane, tetrahydroquinoline, and the like.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

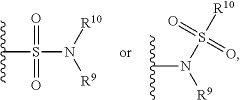

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹

wherein R⁹ represents a hydrocarbyl.

The term “thioether”, as used herein, is equivalent to an ether, wherein the oxygen is replaced with a sulfur.

The term “urea” is art-recognized and may be represented by the general formula

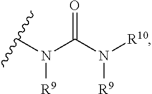

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

### EXAMPLES

The invention now being generally described, it will be more readily understood by reference to the following examples which are included merely for purposes of illustration of certain aspects and embodiments of the present invention, and are not intended to limit the invention.

### Example 1: Screening and Testing of Exemplary Compounds

Targeting Aβ production and/or clearance has, to date, resulted in clinical failure. This is perhaps due to the inability of Aβ-targeted therapy to sufficiently alter the pathogenic events underlying AD, at least under the conditions that these clinical trials were performed. As a result, there is an urgent need to identify new approaches for the treatment of AD. Targeting sAPPα enhancement through modulation of RTN3 is such an approach and will be advantageous, improving cognitive performance while at the same time lowering Aβ levels as cleavage at the a site leads to enhanced sAPPα and is an endogenous inhibitor of BACE1.

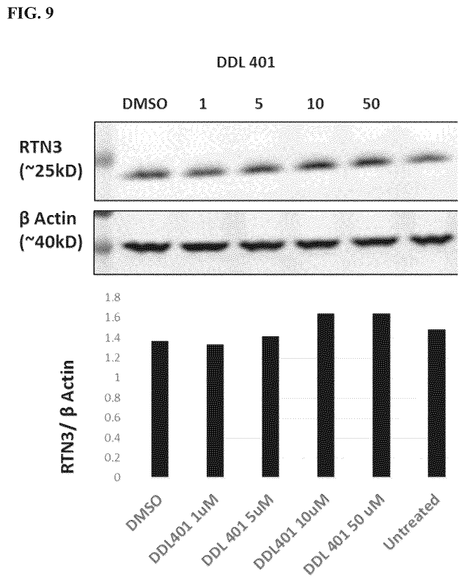

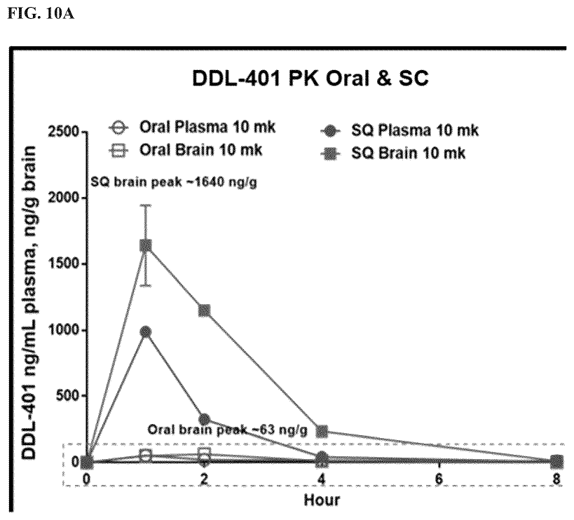

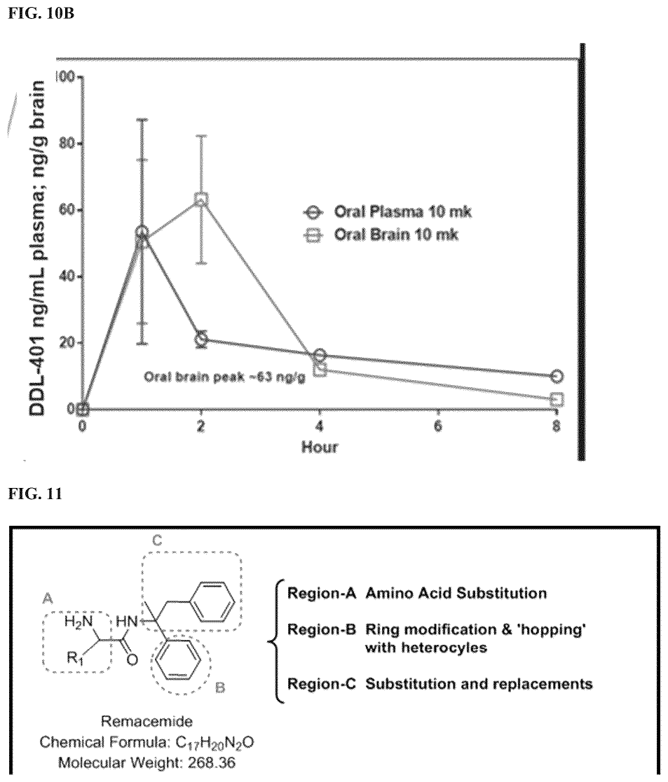

A HTS of portions of the UCLA compound library was undertaken. The primary HTS was carried out at the MSSR facility at UCLA using CHO-7W cells. An initial screen of approximately 7000 compounds was performed using the NIH and NPW libraries, 3 plates from the targeted libraries (TAR), full LOPAC and NKIL libraries, and four plates from the Emerald/Synergy (ES) library. CHO-7W cells (5 K/well) were treated with 5 μM compounds for 48 h followed by collection of media and determination of sAPPα using an AlphaLISA. A scatterplot showing the percent increase in sAPPα levels normalized to control revealed many hits (˜65 hits) that increase sAPPα (≥20%) in HTS screen (FIG. 9). Further validation revealed two compounds that demonstrated sAPPα increase after purchase of dry compound and retest in SH-SY5Y: Remacemide (DDL-401).

DDL-401 elicited an increase in sAPPα of >25% in HTS, and underwent confirmation and secondary screening in CHO-7W and SH-SY5Y cells, respectively. The SH-SY5Y cells (150 K/well) were treated with 1, 5, 10 and 50 μM DDL-401 and DDL-402 for 24 hours. The sAPPα AlphaLISA results showed that DDL-401 significantly (p>0.01) increased sAPPα (FIG. 8).

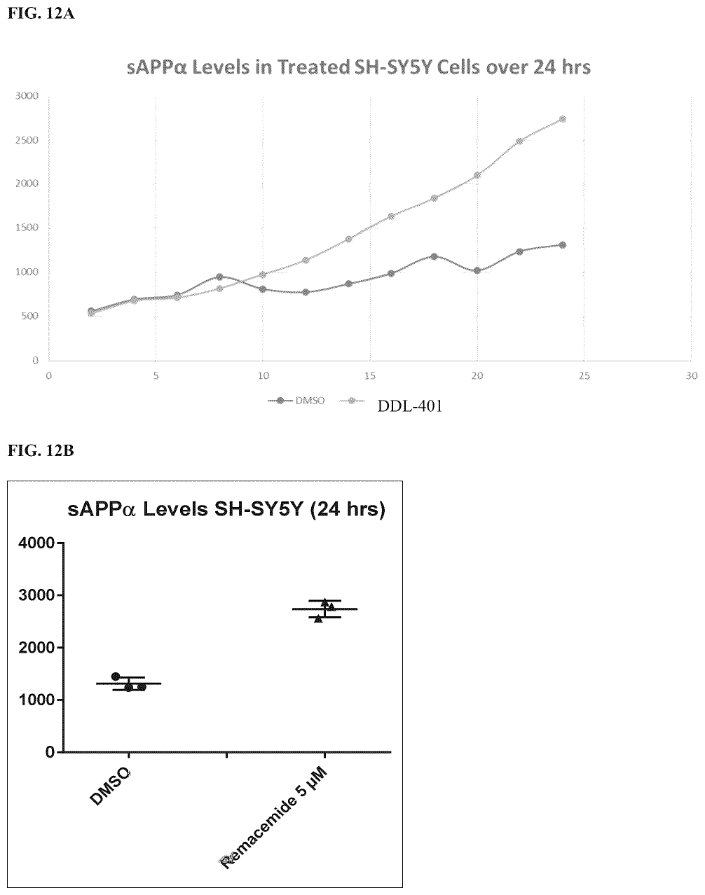

Quantitative global proteomics comparing untreated and DDL-401 treated SH-SY5Y cells were performed to identify the molecular mechanisms underlying DDL-401 sAPPα enhancement. The unbiased nature of this proteomic analysis was particularly attractive as it is also indicative of off target effects. SH-SY5Y cells treated with either DDL-401 or vehicle (DMSO) were lysed, and the proteins were digested with trypsin. Tryptic peptides were subsequently labeled with isobaric tandem mass tags (ThermoFischer Scientific) to provide relative quantitation between samples, and then fractionated using high pH reverse-phase chromatography for increased proteome coverage. The multiplexed samples were analyzed by liquid chromatography tandem mass spectrometry (LC-MS/MS). A series of bioinformatics methods including comprehensive gene set enrichment analysis, pathway analysis, and functional protein association network analysis were utilized to investigate mechanisms underlying DDL-401 mediated sAPPα enhancement. Through global proteomics analysis significant changes in protein abundances in response to DDL-401 treatment (FIG. 12) were identified.

Additional bioinformatics analysis allowed led to the identification of a mechanism of action through which DDL-401 might increase sAPPα. In DDL-401 treated cells, it was observed that an increase in the levels of tryptic peptides in the MS/MS analysis corresponding to the molecular chaperone BAP31, which has been shown to stabilize RTN3 monomers . This led to the hypothesis that the mechanism by which DDL-401 increases sAPPα is through stabilization of RTN3 monomers and possibly through reduction in BACE1 activity, thus making more FL APP substrate available for ADAM10 cleavage (FIG. 7). Furthermore, RTN3 is highly expressed in neurons and variants of RTN3 are a risk factor for AD. This preliminary proteomics study has laid the foundation for our proposal and future proteomics-based approaches for new analogs including quantitative phosphoproteome analysis, thermal proteome profiling, and photoaffinity labeling to further elucidate DDL-401's binding site(s) and mechanism of action.

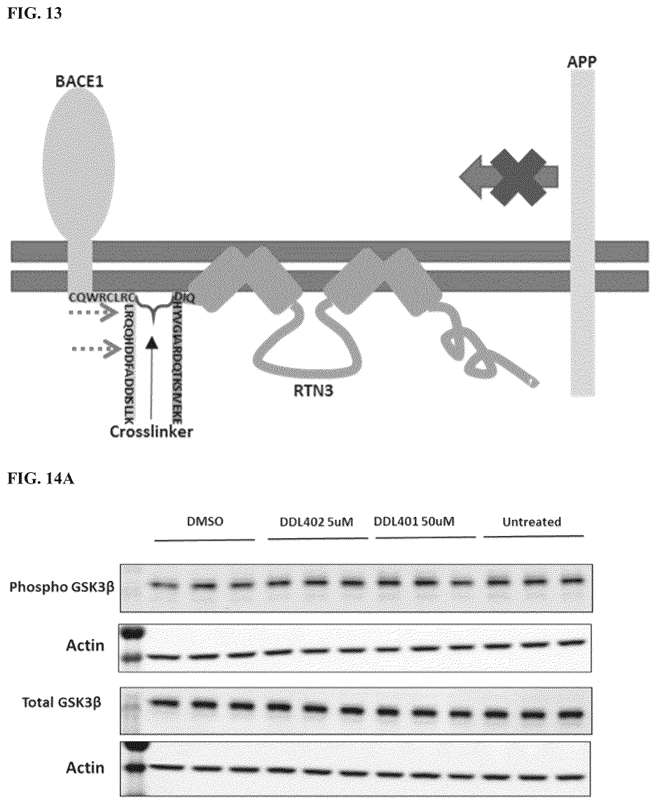

The effect of DDL-401 on the level of RTN3 monomers in vitro in SH-SY5Y cells was analyzed by immunoblot (WB). In response to increasing concentrations of DDL-401 (5, 10 and 50 μM), the level of RTN3 monomers relative to β-actin increased in a dose-response fashion (FIG. 13). Interestingly, no significant increase in BAP31 by WB was seen after DDL-401 treatment. While the preliminary data suggests a modest increase in RTN3 monomer levels at ≥10 uM, the high molecular weight aggregates of RTN3 were not detected in these cells. RTN3-HMW has been shown to be induced in primary neurons.

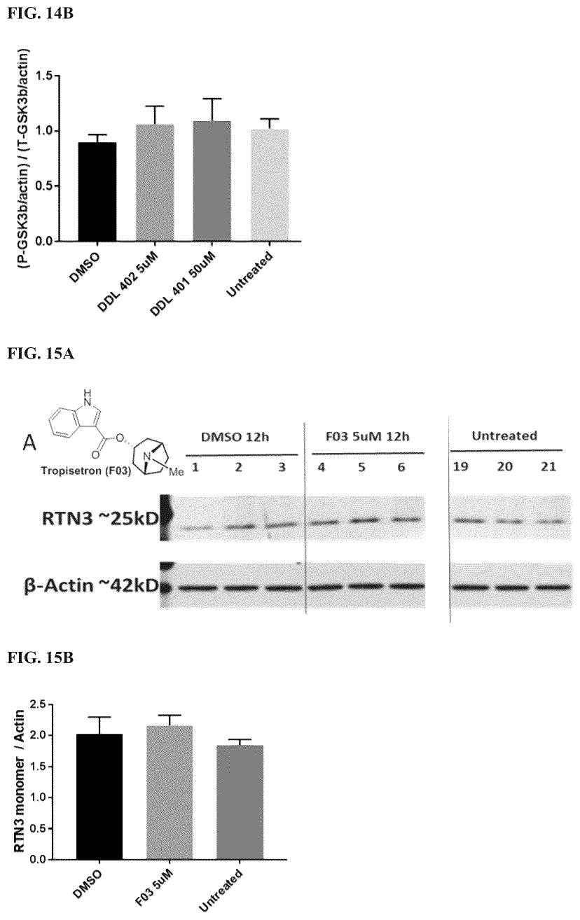

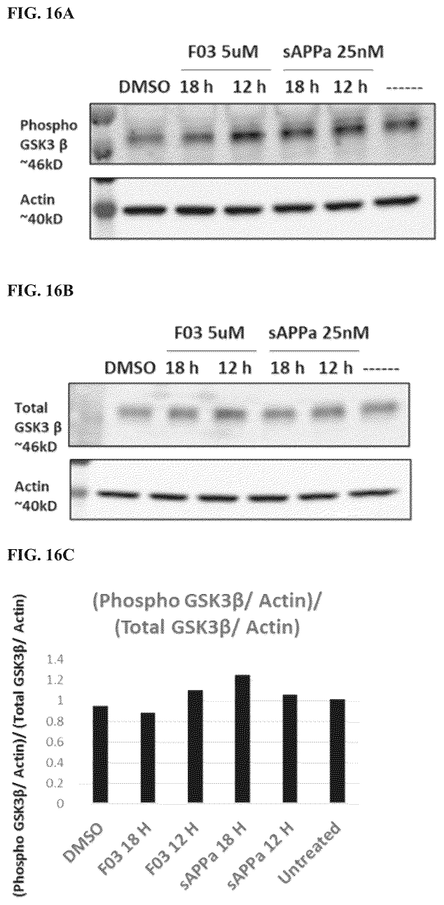

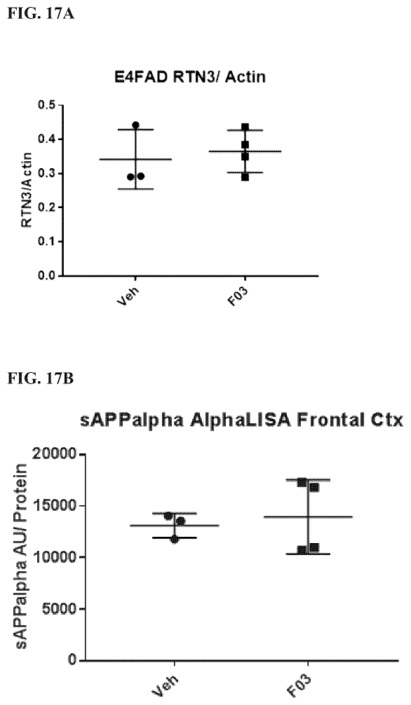

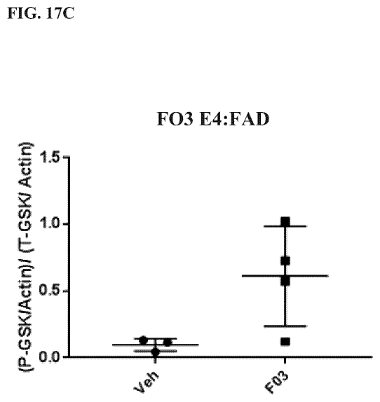

A pharmacokinetics (PK) study to measure the brain levels of DDL-401 using six adult non-transgenic mice injected either subcutaneously (SQ) or dosed orally with DDL-401 in 100% DMSO at 10 mg/kg was performed. Mice were euthanized and perfused with saline 1, 2, 4, and 8 hours later. Brain levels of DDL-401 were determined at Integrated Analytical Solutions (IAS). A brain peak of ˜1640 ng/g was seen 1 hour after SQ injection and of ˜63 ng/g 2 hours after oral delivery (FIG. 14).

### Example 2: Design and Synthesis of Exemplary Compounds

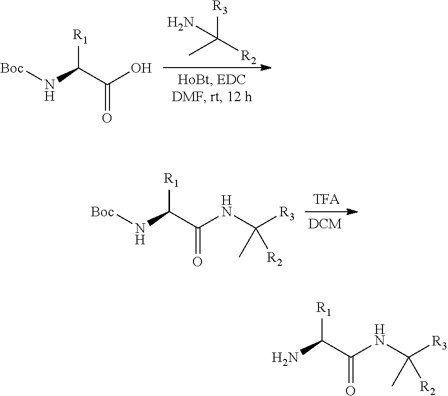

Synthesis of analogs of DDL-401 will be performed using a Syrris flow chemistry microfluidic reactor or by batch chemistry. The synthetic chemistry efforts will focus on drug- like properties, including improved aqueous solubility, microsomal stability, and protein binding (FIG. 11). enable further development of these novel mimetics. The medicinal chemistry strategy to be used to discover new analogs and NCEs is shown in FIG. 11 and Table 1. The outlined strategy is not exhaustive, but simply illustrates the essence of the iterative approach to probe optimization for potency and brain bioavailability.

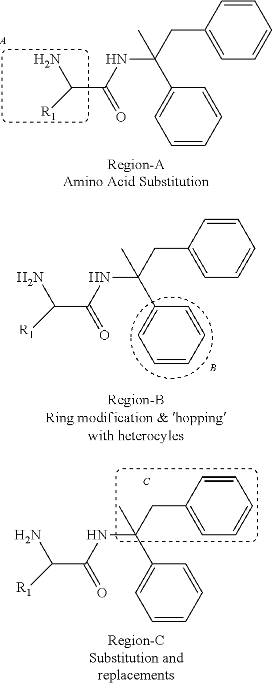

The chemical synthesis of DDL-401 analogs will begin from commercially available Boc-amino acid active-ester that is coupled to the corresponding amines in a batch or flow reactor as shown. For flow chemistry the t-butoxy anhydride of the Boc-amino acid would be prepared (Huang Set al PCT 2006042215, 2006) and used in pump-A while the pump-B will have amines with varying B & C regions. This will be pumped through a preheated glass microfluidic reactor maintained at 40-80° C. (will be optimized) and 2 bar pressure (Syrris Asia Flow Chemistry Module) at a 500 μL/min flow rate with a one-minute residence time in the reactor using Syrris Asia pumps to afford the Boc-DDL-401 analogs from the output stream. After purification of the Boc-derivative by Flash chromatography deprotection would will be performed using TFA/DCM or HCl/dioxane.

### Example 3: Structural Determination of the RTN3-BACE1 Binding Interface by Crosslinking Mass Spectrometry

Chemical crosslinkers will be used to covalently link together amino acid functional groups at adjacent regions of RTN3 and BACE1, while in their native state. After digestion with trypsin, crosslinked peptides will be identified by liquid chromatography tandem mass spectrometry (LC-MS/MS). Crosslinked peptides indicating the binding interface of RTN3 and BACE1 are expected to be near the c-terminus of each protein.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound of formula I or a pharmaceutically acceptable salt thereof: 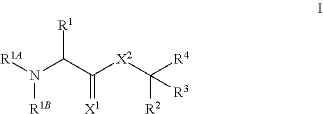
wherein
X¹ and X² are each independently NR^{2A}, O, or S;
R¹ is a side chain of a natural amino acid, alkyl, hydroxyalkyl, alkyloxyalkyl, aminoalkyl, thioalkyl, cycloalkyl, heterocyclyl, aryl, heteroaryl, aralkyl, or heteroarylalkyl;
R² and R⁴ are each independently cycloalkyl, heterocyclyl, aryl, heteroaryl, aralkyl, or heteroarylalkyl;
R³ is H, alkyl, hydroxyalkyl, alkyloxy, alkyloxyalkyl, aminoalkyl, or thioalkyl;
R^{1A}, R^{1B}, and R^{2A} are each independently selected from H, alkyl, and aralkyl; and
provided that the compound is not

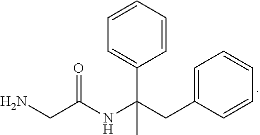 [SMILES: CC(Cc1ccccc1)(NC(=O)CN)c1ccccc1]

2. (canceled)

3. The compound of claim 1, wherein:
1) if X¹ is O, X² is NH, R¹ is H (i.e., the side chain of glycine), R² is phenyl, R³ is methyl, R⁴ is benzyl, and R^{1A} and R^{1B} are both H, then R² is substituted; or
2) if X¹ is O, X² is NH, R¹ is H (i.e., the side chain of glycine), R² is phenyl, R³ is methyl, R⁴ is benzyl, and R^{1A} and R^{1B} are both H, then R⁴ is substituted.

4. (canceled)

5. The compound of claim 1, wherein R¹ is the side chain of alanine, arginine, asparagine, aspartic acid, cysteine, glutamic acid, glutamine, glycine, histidine, isoleucine, leucine, lysine, methionine, phenyl alanine, proline, serine, threonine, tryptophan, tyrosine, or valine.

6. (canceled)

7. The compound of claim 1, wherein X¹ is O.

8. The compound of claim 1, wherein X² is NR^{2A}.

9. The compound of claim 1, wherein R² is aryl or heteroaryl.

10. (canceled)

11. The compound of claim 1, wherein R² is substituted with or more R⁵; and each R⁶ is independently selected from alkyl or halo.

12. The compound of claim 11, wherein R² is substituted with 1, 2, 3, 4, or 5 R⁵.

13. The compound of claim 1, wherein R² is aryl or heteroaryl.

14. (canceled)

15. (canceled)

16. The compound of claim 1, wherein R³ is alkyl.

17. The compound of claim 1, wherein R⁴ is aralkyl, aryl, heteroaryl, or heterocyclyl.

18. (canceled)

19. The compound of claim 1, wherein R⁴ is substituted with or more R⁶; and each R⁶ is independently selected from alkyl, aryl, or halo.

20. The compound of claim 19, wherein R⁴ is substituted with 1, 2, 3, 4, or 5 R⁶.

21. The compound of claim 1, wherein R⁴ is aralkyl, aryl, heteroaryl, or heterocyclyl.

22-24. (canceled)

25. The compound of claim 1, wherein R^{1A} is H.

26. The compound of claim 1, wherein R^{1B} is alkyl or H.

27. (canceled)

28. A pharmaceutically composition comprising a compound of claim 1 and at least one pharmaceutically acceptable excipient.

29. (canceled)

30. A method of treating a neurodegenerative disease or disorder in a subject in need thereof, comprising administering a compound to the subject, wherein the compound is a compound of claim 1, 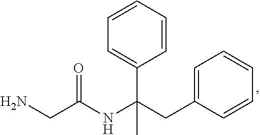 [SMILES: CC(Cc1ccccc1)(NC(=O)CN)c1ccccc1]
or a pharmaceutically acceptable salt thereof.

31. A method of treating a neurodegenerative disease or disorder in a subject in need thereof, comprising administering an upregulator of sAPPα or a stabilizer of reticulon-3 (RTN3) to the subject.

32-37. (canceled)

38. A method of upregulating sAPPα or RTN3 in a cell in vivo comprising contacting the cell with a compound of any one of claims 1-27, 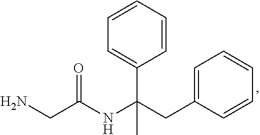 [SMILES: CC(Cc1ccccc1)(NC(=O)CN)c1ccccc1]
or a pharmaceutically acceptable salt thereof.

39-41. (canceled)
